(19) United States
(12) Patent Application Publication
(10) Pub. No.: US 2014/0052050 A1
(43) Pub. Date: Feb. 20, 2014

(54) Illuminated Fabric, Device for Administering Light to the Skin, Kit and Method for Implementing Same

(71) Applicant: Olivier Courtin, Neuilly sur Seine (FR)

(72) Inventor: Olivier Courtin, Neuilly sur Seine (FR)

(73) Assignee: LABORATOIRES CLARINS, Neuilly sur Seine (FR)

(21) Appl. No.: 13/980,776

(22) PCT Filed: Jan. 16, 2012
(86) PCT No.: PCT/IB12/50194

§ 371 Date: Aug. 23, 2013

(30) Foreign Application Priority Data
Jan. 21, 2011 (FR) 1100184

(51) Int. Cl. A61N 5/06; F21V 8/00; F21V 33/00

(52) U.S. Cl. CPC A61N 5/0616; F21V 33/0004; G02B 6/0005
USPC 604/20; 362/556; 607/88

(57) ABSTRACT

The aim of the invention is to provide a device for administering light to the skin, that is cost-effective, reliable, powerful, homogeneous, compact, sufficiently flexible that it stays in contact with the skin of the user, and safe, in that it does not present any risk in the event of excessive or inappropriate use by the user. To this end, the invention relates to an illuminated fabric having a transverse weft direction and a longitudinal warp direction, said fabric comprising, along its length, a plurality of groups of eight consecutive optical fibres interwoven with the textile fibres, each group being transversely woven according to a mixture of Satin 4 weaves, Satin 6 weaves, optionally with floats, and Satin 8 weaves.

The invention relates to an illuminated fabric, to a device for administering light to the skin, and a kit and a method for implementing same.

There are many luminotherapy devices for professional use, that is to say under the control of a professional, such as a doctor or a nurse. This restriction is due to the fact that their incorrect use or use in inappropriate conditions can cause significant damage to the user, ranging from superficial burns to skin cancer through deep burns of the dermis. The user must therefore go to the place of treatment for his or her care.

Such devices are generally very bulky because of the large number of light sources needed to obtain an illumination of the skin that is uniform and powerful enough to obtain a therapeutic effect of the light on the skin.

The light sources used in these professional devices are generally of two main types: light tubes and light diodes. As an example, the GentleWaves Pro Unit or Aktilite CL128 appliance can be cited.

Solutions have been proposed that make it possible to reduce the bulk of these devices.

For example, a mask made of rigid plastic has been proposed that incorporates light-emitting diodes. However, this device is uncomfortable to wear and has to be adapted to each user because of his or her morphology and the rigidity of the mask. Furthermore, tests have shown that the uniformity of the light was very poor: the light exhibits a maximum intensity in line with the diodes and loses its intensity with distance from the diodes. To improve the uniformity, one obvious solution is to increase the number of diodes. However, it is found that this solution is much more complex than it would seem to obtain a satisfactory uniformity.

Furthermore, the system for supplying each diode with electricity and the heat evacuation system are unattractive and still remain rigid and too bulky.

Another solution that has been envisaged was to create an illuminated fabric based on optical fibers with at least one of the ends linked to a light source.

Such a fabric is described in the document WO 2006/031350.

This fabric offers the advantage of being less rigid than the masks made of plastic, and of being less bulky, because the light source can be relocated and takes less space than a diode circuit and its power supply.

Nevertheless, measurements have shown that the light energy emitted by such fabrics is inadequate and non-uniform.

To remedy the problem of power, it has naturally been proposed to increase the power of the light source or sources or increase their number.

However, this increases the energy consumption as well as the bulk.

Furthermore, it has been proposed to illuminate both ends of each fiber. However, each source receives, via the optical fibers, light energy from the other source. This poses a significant problem in the case of light diodes, in particular laser diodes, because they risk being rapidly and definitively damaged.

To remedy the problem of uniformity, it has been proposed to increase the number of optical fibers.

However, this increases the device, its rigidity, its fragility, and provides only a very modest improvement to the uniformity of the light emitted.

A first objective of the present invention is to propose a device for administering light to the skin that is cost-effective, reliable, powerful, uniform, not bulky, comfortable to wear and that can easily be adapted to any user, and that is sufficiently flexible to remain in contact with the skin of the user.

Moreover, it has been observed that the administration of the light to the skin could have an anti-aging and soothing esthetic effect, in particular on the face.

To facilitate the use of light for an esthetic purpose, there is therefore a need for a device for administering light to the skin that allows for personal use, that is to say whose use does not necessarily require the control of a professional. With such a device, the user can choose to provide esthetic care in the home, without having to go to a beauty institute or to a doctor.

A second objective of the invention is to propose a device for administering light to the skin that is both effective and safe, i.e. that does not present any danger if used excessively or inappropriately by the user.

To this end, the subject of the invention is an illuminated fabric having a transversal weft direction and a longitudinal warp direction, characterized in that it comprises, longitudinally, a plurality of groups of eight consecutive optical fibers interwoven with textile fibers, each group being interwoven transversely according to a mixture of satin 4 weaves, of satin 6 weaves, possibly with floats, and of satin 8 weaves.

According to other embodiments:
the illuminated fabric may also comprise additional optical fibers interwoven transversely according to a mixture of incomplete satin 4, satin 6 and satin 8 weaves;
the illuminated fabric may exhibit the following features:
   each group is interwoven transversely according to an alternation of patterns A made up of a satin 8 weave and of patterns C made up of four satin 4 weaves arranged in a square, the patterns A and C being systematically separated by a pattern B made up of a satin 6 weave, possibly with float; and
   two longitudinally successive groups have weaves A and C alternated in opposition;
an eighth to a quarter of the satin 6 weave may consist of float lines in which the optical fibers are not interwoven;
the illuminated fabric may exhibit the following features:
   the pattern A has a transversal dimension of between 40 and 50 millimeters, advantageously 44 millimeters;
   the pattern B has a transversal dimension of between 25 and 35 millimeters, advantageously 30 millimeters;
   the pattern C has a transversal dimension of between 55 and 70 millimeters, advantageously 62 millimeters;
the illuminated fabric may have a density of optical fibers of between 29 and 40 optical fibers per cm;
the optical fibers may have a core of PMMA and a sheath of fluorinated polymer;
the sheath may have a thickness of 5 μm, the optical fibers having an overall diameter of 250 μm;
the textile fibers may be made of a material chosen from polyester, cotton, linen, polypropylene, polyamides, or a mixture thereof;
the illuminated fabric may have a flexibility of less than or equal to 2000 mN, the flexibility being defined as being the force needed to bend a 3.5 cm×5.0 cm sample by 30°.

The invention also relates to an illumination device intended to be arranged in contact with the skin of a user, comprising at least one light source and one illuminated fabric according to the preceding invention, in which at least one end of each optical fiber is connected to a light source.

According to other embodiments:
the light source may have a wavelength of between 440 nm and 680 nm, preferably between 440 nm and 500 nm, and/or between 590 nm and 680 nm;
the device may also comprise a means for storing the duration and/or the frequency of use and a timer capable of stopping or disabling the operation of the light source(s);
the device may also comprise a means for reading operating information concerning the device, the reading means being functionally linked with a central processing unit capable of controlling or disabling the operation of the device according to the operating information.

The invention also relates to a cosmetic skin treatment kit, comprising a device according to the preceding invention and at least one cosmetic product container, the container bearing operating information concerning the device.

According to other embodiments:
the container may comprise an RFID chip on which is stored usage information, the reading means of the device being an RFID chip reader;
the container may comprise a one- or two-dimensional barcode graphically representing the usage information, the reading means of the device being a one- or two-dimensional barcode reader.

The invention also relates to a method for implementing a kit according to the preceding invention, the method comprising the following steps:
implementing the reading means to read the operating information and activate the device in accordance with this information;
directly applying the illuminated fabric to the skin of a mammal for illumino-cosmetic treatment of the skin of the mammal;
   a duration and a mode of operation of the light source being automatically controlled according to the operating information.

According to other embodiments:
the cosmetic product may comprise at least one active product taken from the group made up of: palmitoyl dipeptide 5 diaminohydroxybutyrate, extract of Cochlearia officinalis, a ferment of Thermus thermophillus, tiliroside, pyroglutamylamylamidoethyl indole, an association of tiliroside/palmitoyl dipeptide 5 diamonohydroxybutyrate, extract of Rhodiola rosea, extract of Spergularia rubra, extract of Camellia sinensis, extract of Lapsana communis, extract of Atractyloides lancea, extract of Cedrelopsis gravei, extract of Thymus citriodorus, Chrysine, extract of Dianthus carthusianorum, or a mixture thereof;
the product may be applied before or after the application of the illuminated fabric to the skin.

Other features of the invention will emerge from the following detailed description, given with reference to the appended figures which represent, respectively:

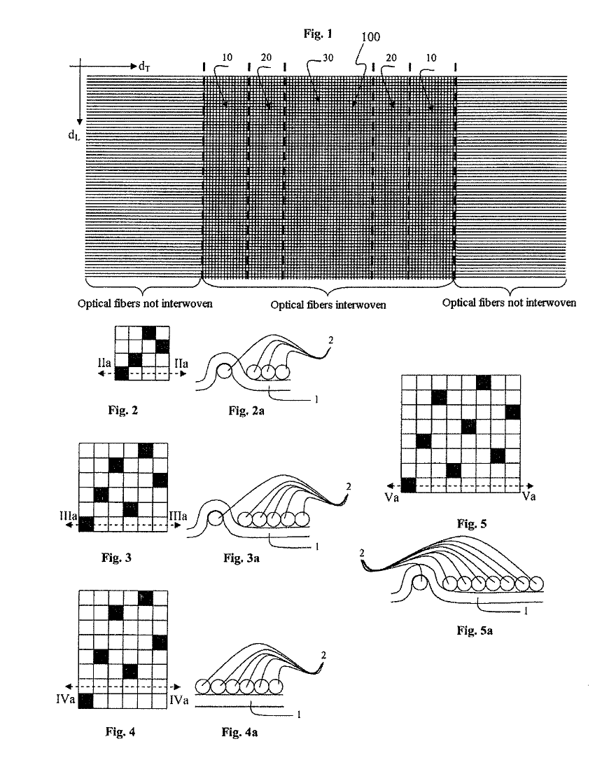

FIG. 1, a schematic plan view of an illuminated fabric according to the invention;

FIGS. 2 to 5, schematic plan view representations of weaves, respectively, of satin 4, of satin 6, of stain 6 with float and of satin 8;

FIGS. 2a to 5a, partial schematic views in cross section of weaves, respectively, of satin 4, of satin 6, of satin 6 with float and of satin 8;

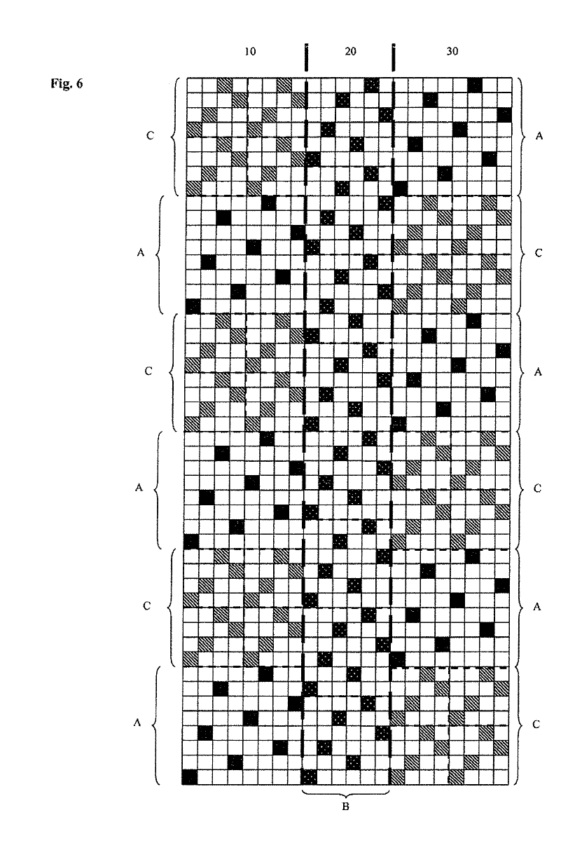

FIG. 6, a schematic plan view representation of a first embodiment of a woven assembly intended for part of an illuminated fabric according to the invention;

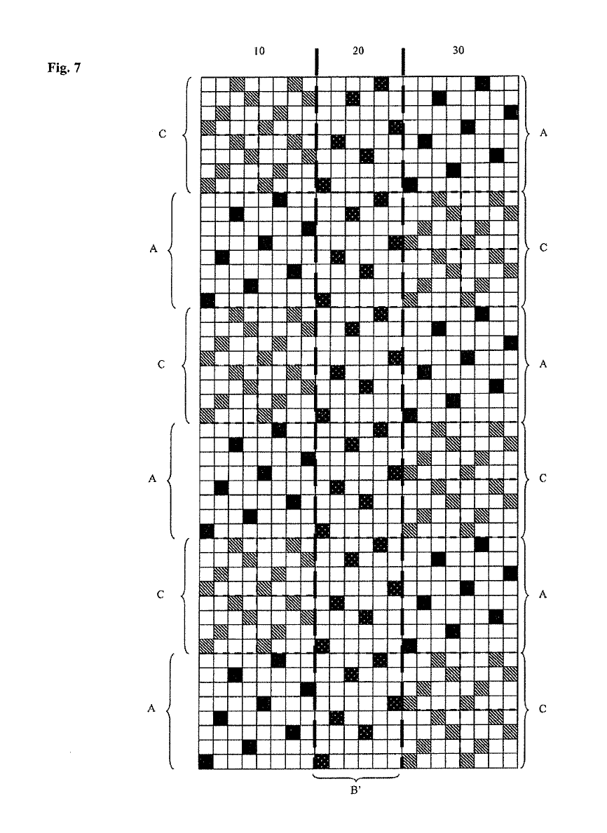

FIG. 7, a schematic plan representation of a second embodiment of a woven assembly intended for part of an illuminated fabric according to the invention;

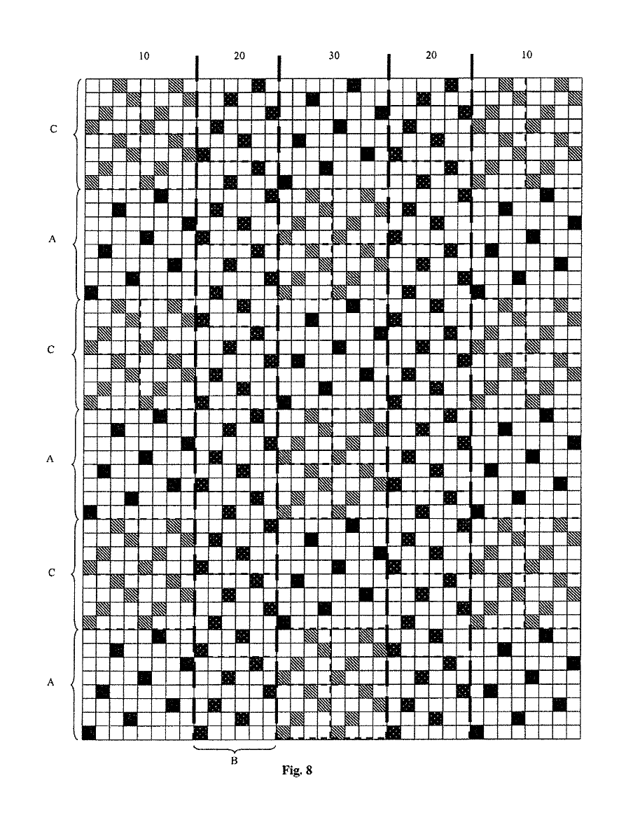

FIG. 8, a schematic plan view representation of a third embodiment of a woven assembly intended to form part of an illuminated fabric according to the invention;

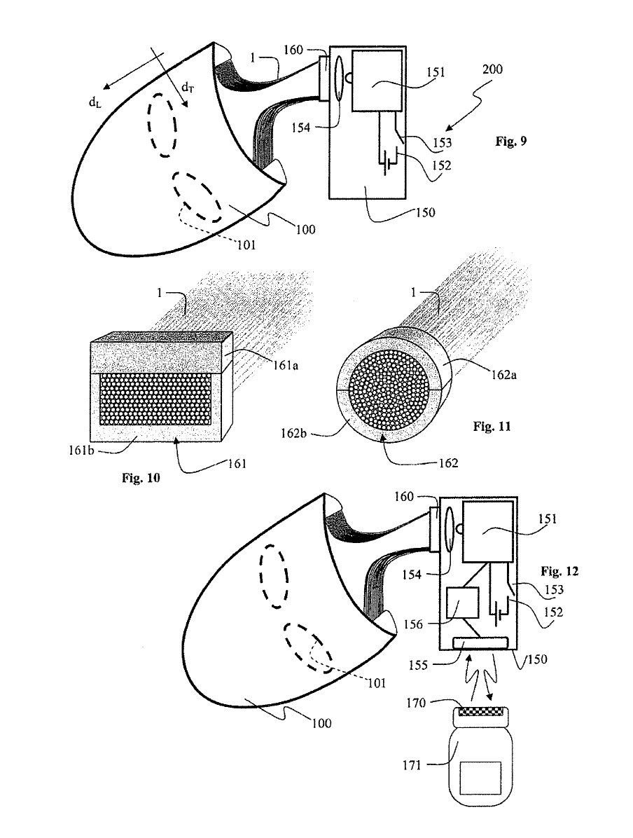

FIG. 9, a schematic representation of a first embodiment of a light device according to the invention;

FIGS. 10 and 11, schematic front views of two embodiments of optical fiber connectors in a light device according to the invention; and

FIG. 12, a schematic representation of a second embodiment of a light device according to the invention incorporated in a kit comprising a container of cosmetic product.

The invention relates to an illuminated fabric and, in particular, to an illuminated fabric intended to be used in application against the skin of a user.

This illuminated fabric 100 is, as illustrated in FIG. 1, made up, along a longitudinal direction d_{L} of the fabric, of warp threads of textile fibers and, along a transversal direction d_{T} of the fabric, of weft threads of optical fibers.

In the transversal direction d_{T}, the illuminated fabric according to the invention comprises an alternation of strips 10-20-30 that are differentiated by the way they are woven.

More particularly, the fabric according to the invention comprises, longitudinally, a plurality of groups of eight consecutive optical fibers interwoven with textile fibers, each group being interwoven transversely according to a mixture of satin 4 weaves, of satin 6 weaves, possibly with floats, and of satin 8 weaves. The representation of these satin 4, satin 6, satin 8 weaves are illustrated respectively in FIGS. 2, 3 and 5.

In this type of representation that is well known to the person skilled in the art, the white cells represent the warp textile fibers whereas the black cells represent the weft threads of optical fiber.

A satin 6 weave with float means that certain optical fibers of the satin 6 weave are not interwoven with the textile fibers. A satin 6 weave with float is illustrated in FIG. 4 where it is possible to see, compared to the satin 6 weave of FIG. 3, two totally white lines, that is to say lines without interweaving of the optical fiber with the textile fibers.

Thus, at least two optical fibers of each group of eight optical fibers are not interwoven in the satin 6 weave with float and pass directly from the pattern A of satin 8, respectively C of satin 4, to the pattern C of satin 4, respectively A of satin 8.

FIGS. 2a, 3a, 4a and 5a respectively illustrate the satin 4, satin 6, satin 6 with float and satin 8 weaves in cross section according to the arrows IIa-IIa, IIIa-IIIa, IVa-IVa and Va-Va.

Thus, in the satin 4 weave (FIG. 2a), the optical fiber 1 passes over a textile fiber 2 (corresponds to the black cell of the checkerboard of FIG. 2) and under three textile fibers 2 (corresponds to the white cells of the checkerboard of FIG. 2).

Advantageously, the satin 4 weave used has a rough appearance by virtue of an irregular weft effect (vertical counter value of +1; +2; −1; −2, see FIG. 2).

In the satin 6 weave (FIG. 3a), the optical fiber 1 passes over a textile fiber 2 (corresponds to the black cell of the checkerboard of FIG. 3) and under five textile fibers 2 (corresponds to the white cells of the checkerboard of FIG. 3).

Advantageously, the satin 6 weave used has a bright appearance, by virtue of an irregular weft effect (vertical counter value of +2; +2; −3; +4; −2; −3; see FIG. 3).

The same applies for the satin 8 weave (FIG. 5a), where the optical fiber 1 passes over a textile fiber 2 (corresponds to the black cell of the checkerboard of FIG. 5) and under seven textile fibers 2 (corresponds to the white cells of the checkerboard of FIG. 5).

Advantageously, the satin 8 weave used has a bright appearance, by virtue of a weft effect with vertical counter of 3 (see FIG. 5).

For the satin 6 weave with float, FIG. 4a illustrates a cross section at the level of a float line. The optical fiber 1 therefore passes under all the textile fibers 2, which explains the white cells representing the float line.

The general weave of the fabric is shown in FIG. 6, the patterns A, B and C being filled by, respectively, satin of 4 irregular weft effect (vertical counter value of +1; +2; −1; −2), satin of 6 irregular weft effect (vertical counter value of +2; +2; −3; +4; −2; −3) and satin of 8 weft effect with vertical counter of 3. For this weave, the weft ratio is 48 (smaller common product between a weave with a weft ratio of 6 and a weave with a weft ratio of 8).

In other words, the illuminated fabric according to the invention comprises, longitudinally, a plurality of groups of eight consecutive optical fibers, each group being interwoven transversely according to an alternation of patterns A made up of a satin 8 weave and of patterns C made up of four satin 4 weaves arranged in a square, the patterns A and C being systematically separated by a pattern B made up of a satin 6 weave, possibly with float (denoted B′ hereinbelow).

Furthermore, longitudinally, two successive groups have weaves A and C alternated in opposition.

Obviously, the number of weaves A, B or B′ and C is preferably calculated to obtain a fabric that has a substantially rectangular form. In other words, to interweave eight consecutive optical fibers according to a satin 4 weave, at least two repetitions are needed in the transversal direction d_{T} of the satin 4 weave and two repetitions are needed in the longitudinal direction d_{L}. This is illustrated in FIG. 6 where it is possible to see, in the strip 10, a first pattern C made up of four satin 4 weaves, corresponding to the interweaving of eight optical fibers, followed, in the longitudinal direction d_{L}, by a pattern A made up of a satin 8 weave.

In FIGS. 6 to 8, the broken lines are used only to facilitate the distinction between the different weaves, and do not correspond to any physical reality in the illuminated fabric according to the invention. Furthermore, the cells representing the visible optical fibers are not all black, since they would normally have had to be represented according to the usual practice, but they represent different patterns only intended to facilitate the identification of the different weaves and patterns. Thus, the interwoven optical fibers in satin 4 are illustrated by cells with cross-hatching, the interwoven optical fibers in satin 6 are illustrated by black cells with white dots, and the interwoven optical fibers in satin 8 are illustrated by black cells.

The fabric illustrated in FIG. 6 therefore comprises an assembly made up of:
a first strip 10 comprising, in the longitudinal direction d_{L}, three patterns A each made up of a satin 8 weave and three patterns C each made up of four satin 4 weaves arranged in a square, the patterns A and C being alternated;
a second strip 20 comprising, in the longitudinal direction d_{L}, eight patterns B each made up of a satin 6 weave; and
a third strip 30 comprising, in the longitudinal direction d_{L}, three patterns C each made up of four satin 4 weaves arranged in a square and three patterns A each made up of a satin 8 weave, the patterns C and A being alternated. Furthermore, when the first strip has a pattern A, the third strip has, transversely, a pattern C. The patterns A and C are therefore alternated in opposition.

The illuminated fabric according to the invention advantageously has a plurality of above assemblies in the longitudinal direction d_{L}.

FIG. 7 illustrates a variant of the assembly of FIG. 6, in which the strip 20 comprises six patterns B′ each made up of a satin 6 weave with float.

The warp threads used are, for example, made of a white 100% polyester thread, of 33 Tex made up of 288 filaments, produced by Sinterama under the reference T371.

The weft threads used are of optical fibers, for example, made of polymethylmethacrylate (PMMA), with a diameter of 250 μm (58 Tex), produced by Toray under the reference PGR-FB250. The diameter cited comprises a fluorinated sheath 5 μm thick.

The width (in the transversal direction) of the patterns depends on the density of warp threads. The length (in the longitudinal direction) of the patterns depends on the density of weft threads.

In the exemplary embodiment, the density of warp threads is set at 20 threads/cm.

Different weft densities have been produced, ranging from 29 fibers/cm to 40 fibers/cm (ceiling limit set for geometrical reasons). The most interesting results have been obtained for densities around 37 fibers/cm (35 to 40 fibers/cm).

Given the morphology and the general size of the faces, an illuminated fabric according to the invention used as mask needs to have a width (in the transversal direction) greater than or equal to 200 mm on the loom. To meet this dimension and given the density of warp threads (20 threads/cm), the fabric according to the invention comprises two additional strips 10 and 20 compared to the embodiment of FIGS. 6 and 7.

The best results in terms of uniformity of the light diffused are obtained with a fabric comprising at least one assembly illustrated in FIG. 8 and made up of:
a first strip 10 comprising, in the longitudinal direction d_{L}, three patterns A each made up of a satin 8 weave and three patterns C each made up of four satin 4 weaves arranged in a square, the patterns A and C being alternated;
a second strip 20 comprising, in the longitudinal direction d_{L}, eight patterns B each made up of a satin 6 weave; and
a third strip 30 comprising, in the longitudinal direction d_{L}, three patterns C each made up of four satin 4 weaves arranged in a square and three patterns A each made up of a satin 8 weave, the patterns C and A being alternated. Furthermore, when the first strip has a pattern A, the third strip has, transversely, a pattern C. The patterns A and C are therefore alternated in opposition;
a fourth strip 20 comprising, in the longitudinal direction d_{L}, eight patterns B each made up of a satin 6 weave; and
a fifth strip 10 comprising, in the longitudinal direction d_{L}, three patterns A each made up of a satin 8 weave and three patterns C each made up of four satin 4 weaves arranged in a square, the patterns A and C being alternated as in the first strip.

Alternatively, the second and fourth strips 20 can comprise patterns B′ each made up of a satin 6 weave with float.

Thus, each optical fiber alternates an interweaving of satin 4 or of satin 8/an interweaving of satin 6 or of satin 6 with float/an interweaving of satin 8 or satin 4.

This sequence of strips 10-20-30-20-10 makes it possible to obtain, at one and the same time, an illuminated fabric that can be relatively wide and flexible, and a very good uniformity of diffusion of the light.

To produce a light mask according to the invention, a fabric is produced in which:
the pattern A has a width (in the transversal direction) of between 40 and 50 millimeters, advantageously 44 millimeters;
the pattern B has a width (in the transversal direction) of between 25 and 35 millimeters, advantageously 30 millimeters;
the pattern C has a width (in the transversal direction) of between 55 and 70 millimeters, advantageously 62 millimeters.

Below are indicated (table 1), for each longitudinal strip, the width, the number of threads of the pattern, the number of repeats of the pattern and the total number of threads.

| | Strip | Strip | Strip | Strip | Strip | |
|---|---|---|---|---|---|---|
| | 10 | 20 | 30 | 20 | 10 | Total |
| Width (mm) | 44 | 30 | 64 | 30 | 44 | 212 |
| Number of | 8 | 6 | 8 | 6 | 8 | |
| threads of | | | | | | |
| the pattern | | | | | | |
| Number of | 11 | 10 | 16 | 10 | 11 | |
| repeats of | | | | | | |
| the pattern | | | | | | |
| Total number | 88 | 60 | 128 | 60 | 88 | 424 |
| of threads | | | | | | |

In all, the fabric according to FIG. 8 can therefore reach a transversal dimension of 20 or so centimeters (21.2 in the preceding exemplary embodiment).

The overall width of the fabric comprises 424 threads for a machine width of 212 mm on the loom.

Along the lateral edges of the strips 10, two gas stitch interwoven threads are introduced in order to prevent the de-interweaving of the structure.

To be able to be connected to the light source, the optical fibers, introduced in the weft, are cut at approximately 290 mm from the edges of the fabric. For easy handling of the product to be connected, they are held by a sail stitch (or any other weaves) at approximately 20 mm from their ends. The overall width of the product to be connected is approximately 792 mm.

Advantageously, the density of optical fibers is between 29 and 40 optical fibers per centimeter in the longitudinal direction d_{L}, preferably 37 fibers/cm.

To produce a mask, a choice is made to manufacture an illuminated fabric that has at least 20 or so centimeters in the longitudinal direction d_{L}. More specifically, in the exemplary embodiment described previously, the fabric has a length of 22 cm.

This corresponds to 816 picks of optical fiber or 17 repeats of a weft weave ratio of 48 picks (for an optical fiber density of 37 threads/cm).

Before and after the interweaving of the optical fibers, a textile thread is advantageously interwoven to prevent the de-interweaving of the optical fibers from the ends and improve the general visual appearance of the fabric.

This fabric has been obtained produced by using a comb containing 5 cells per centimeter. For each of the cells, 4 threads are introduced.

In all, 24 frames were used:
8 are used to interweave the longitudinal strips 10 (8 threads for the pattern repeated 11 times).
6 are used to interweave the longitudinal strips 20 (6 threads for the pattern repeated 10 times).
8 are used to interweave the longitudinal strip 30 (8 threads for the pattern repeated 16 times).
2 are used to produce the sail stitch (or other weave that can produced on two frames) used to hold the optical fibers.

A protection 101 (see FIGS. 9 and 12) of the sensitive areas, such as the eyes, is advantageously provided on the front of the fabric intended to be arranged on the face. This protection must be opaque to the wavelengths emitted by the illuminated fabric.

The light uniformity of the fabric according to the invention is measured as follows.

A light source is linked to the two ends of each optical fiber. For example, it is possible to use a light-emitting diode (LED) delivering a light power of 1.32 W at a wavelength of 630 nm.

Then, a measurement of the light intensity (IL) is carried out on a surface of 1 cm*1 cm using a wattmeter. For example, a wattmeter can be used with a 1 cm² probe of Ophir brand name and model PD300. The measurement is stored.

The measurement of IL is reiterated at a number of points on the illuminated fabric, advantageously over the entire surface of the fabric.

The measurements are then compared with one another to calculate the standard deviation between these measurements, this deviation being expressed as a percentage.

Uniformity is represented by this standard deviation.

The fabric according to the invention has a standard deviation less than 30% and substantially equal to 25% (to within plus or minus 3%).

The illuminated fabric according to the invention also has a very good flexibility making it possible both to fold it for storage purposes, and to spread it out on the face for it to cover the face in a relatively uniform manner. Obviously, the illuminated fabric does not completely follow the form of the face because of the protruding points such as the nose or the mouth, but it comes into contact with most of the surfaces of the face. It is therefore totally differentiated from the illuminated fabrics bonded in resin or other polymer, such as the fabrics provided for the treatment of infant jaundice and flat, between a support table and the infant placed above.

There is no normative method for measuring the flexibility of a fabric. In order to quantify the flexibility of the fabric obtained according to the invention, measurements have been carried out with a methodology used to measure the flexibility of cardboards: this involves measuring the force (in newtons) needed to bend the sample (3.5×5.0 cm) by an angle of 30°. The results of the measurements performed on a sample of fabric according to the invention show that the force needed remains less than or equal to 2000 mN, including for a number of optical fibers per centimeter greater than 30. This measurement was carried out with a “Bachel stiffness tester” machine.

By virtue of the interweaving according to the invention, a very flexible illuminated fabric is obtained, that is to say one that requires a force less than or equal to 2000 mN to bend a reference sample by 30°.

Advantageously, the optical fibers have a core of PMMA and a sheath of fluorinated polymer. Advantageously, the optical fibers used have a sheath that has a thickness of 5 μm and have an overall diameter (sheath+core) of 250 μm. This type of fiber gives excellent light conduction and lateral light transmission results by virtue of the interweaving.

In practice, by virtue of the interweaving according to the invention, it is pointless to mechanically create defects or imperfections in the sheath to extract the light laterally. The optical fibers according to the invention are therefore made without such defects or imperfections that make it possible, by virtue of the particular interweaving of the invention, to emit light laterally through the sheath, and uniformly so that the illumination is uniform.

Furthermore, the interweaving according to the invention avoids having light emitted at one end being introduced into the light source at the other end. Thus, the light source does not heat up and its lifespan is optimized.

The textile fibers used can be of different materials. However, to ensure that the fabric has a mechanical strength and resistance, while ensuring its flexibility and its softness (it is recalled that this fabric is intended to be placed on the face of a user), the textile fibers are advantageously chosen to be of polyester. Other materials chosen from among cotton, linen, polypropylene, polyamides, or a mixture thereof, etc., can also be used.

Regarding the tension of the warp threads, it is essential to find a happy medium for bending the optical fibers and extracting the light all along the fibers without the lights being reinjected at the opposite end to avoid reintroducing light into the light source or sources. In practice, if, during the interweaving, the warp threads are not taut enough, light is transmitted to the opposite end and risks damaging the light source. If the warp threads are too taut, the optical fibers are very bent and the very great majority of the light risks exiting through the edges of the fabric while the center of the fabric will emit only a very small amount of light, thus affecting the uniformity of the light emission.

The optical fibers can be grouped together according to determined spatial distributions. For example, the fibers intended to cover the surround of the eye are grouped together, the fibers intended to administer light to the forehead are grouped together, and so on. The device can then comprise a means for selectively lighting the ends of the groups of fibers in order to administer or not administer light to one or more particular areas, but also to modulate the lighting power according to the area to be lit by having, for example, filters between the light source or sources and the ends of the groups of fibers. It is thus possible to light certain areas selectively and in a controlled manner relative to other areas. However, by virtue of the interweaving according to the invention, each area exhibits uniform illumination, even if the value of the illumination may vary from one area to the other in a controlled manner.

The illuminated fabric according to the invention may also comprise additional optical fibers interwoven transversely according to a mixture of incomplete satin 4, satin 6 and satin 8 weaves. In other words, between two transversal groups of eight optical fibers interwoven according to complete satin 4, satin 6 and satin 8 weaves, the fabric may comprise incomplete weaves, in the longitudinal direction, of satin 4, satin 6 and satin 8.

The illuminated fabric according to the invention is advantageously intended to be incorporated in a device for administering light to the skin of a user, the device comprising at least one light source and an illuminated fabric described previously according to the invention, at least one end of each optical fiber being connected to a light source.

Advantageously, to limit the bulk of the device, the two ends of each fiber are connected to one and the same light source.

The light source advantageously has a wavelength of between 440 and 680 nm, preferably between 440 nm and 500 nm, for example for the treatment of so-called skins said to be “with imperfections” (fat skin), and/or between 590 nm and 680 nm to obtain an anti-aging and soothing effect.

It is thus advantageously possible to use a combination of two wavelengths such as, for example, red and infrared.

Such a device is illustrated in FIG. 9.

In this figure, the device 200 comprises a fabric 100 illustrated in the form of a mask, of which the optical fibers 1 are linked to a box 150 provided with a light source 151 so that the ends of each fiber are arranged facing the light source 151. It is also possible to use a plurality of light sources (such as diodes) in the same box.

The light source is electrically linked to an energy source 152 and to a switch 153 to switch the light source 151 on and off. The latter can operate continuously or in pulse mode, for example according to one of the following sequences:
cycles of 250 milliseconds on/100 milliseconds off, 100 cycles for 35 seconds of exposure; or
cycles of 500 microseconds on/150 microseconds off, four cycles per sequence, 750 microseconds or 100 milliseconds of intervals between two sequences for 1000 seconds of exposure.

An optical lens 154 can advantageously be inserted between the source and the optical fibers to concentrate the light on said ends and limit the light transmission losses.

The link between the ends of optical fibers and the box 150 is made by a connector 160 of which two embodiments 161 and 162 are illustrated in FIGS. 10 and 11.

These two embodiments are differentiated by the form given to all the ends of the optical fibers grouped together: rectangular (FIG. 10) or circular (FIG. 11). The latter form offers the advantage of limiting the light transmission losses and favoring the uniformity of the light transmission by the fabric because all the ends are illuminated uniformly and all the light emitted by the source 151 is sent to the fibers. The lens 153 also plays an important role in this optimal light transmission.

The connectors illustrated in FIGS. 10 and 11 have two parts 161a-162a and 161b-162b which form jaws gripping the optical fibers 1 so as to limit the space between the optical fibers 1.

To improve the light transmission, the ends of the optical fibers are buffed.

Advantageously, the device according to the invention also comprises a means for storing the duration and/or the frequency of use and a timer capable of stopping or disabling the operation of the light source(s).

It is thus possible to limit the duration and/or frequency of use of the device for administering light to the skin according to the invention, which allows use by the general public, without the supervision of a doctor.

According to a particularly advantageous embodiment, the device according to the invention forms part of a kit and also comprises a means 155 for reading operating information 170 concerning the device, the reading means 155 being functionally linked with a central processing unit 156 capable of controlling or disabling the operation of the device according to the operating information.

The central processing unit 156 may incorporate the storage means and the timer.

The operating information 170 may be borne by a container 171 of cosmetic product, such as a pot or tube, included in the kit. This information 170 should be in a form that can be read and interpreted automatically by the reader, for example in the form of a one- or two-dimensional barcode graphically representing the usage information, an RFID chip on which is stored the usage information, and so on.

The reading means 155 of the device is then chosen from a one- or two-dimensional barcode reader, an RFID chip reader, and so on.

This reading means 155 is linked to the central processing unit 156 so as to control or disable the operation of the device according to:
the information read on the pot or the tube of cosmetic product; and/or
the duration of use of the device.

The information may be an operating authorization or interdiction, but also an operating instruction comprising, for example, the maximum exposure time, operation in continuous mode or in pulsed form, a determined spatial distribution of the light power, etc.

To implement the kit according to the invention, the following steps are implemented:
activating the reading means 155 to read the operating information 170 and activate the device (light source and illuminated fabric) in accordance with this information. This activation is done via the central processing unit 156;
directly applying the illuminated fabric 100 to the skin of a mammal for a lumino-cosmetic treatment of the skin of the mammal;
a duration and a mode of operation of the light source being automatically controlled according to the operating information.

Other settings of the device may also be provided, such as, for example, the age of the user, his or her skin type, the frequency of use, etc.

By virtue of the kit according to the invention, it is possible to operate the illuminated fabric in a manner suited to the cosmetic product used and to the user. Furthermore, the latter cannot use the illuminated fabric in a dangerous manner, so that use by the general public is possible.

Advantageously, the cosmetic product comprises at least one active ingredient taken from the group consisting of: palmitoyl dipeptide 5 diaminohydroxybutyrate, extract of Cochlearia officinalis, a ferment of Thermus thermophillus, tiliroside, pyroglutamylamylamidoethyl indole, an association of tiliroside/palmitoyl dipeptide 5 diaminohydroxybutyrate, extract of Rhodiola rosea, extract of Spergularia rubra, extract of Camellia sinensis, extract of Lapsana communis, extract of Atractyloides lancea, extract of Cedrelopsis gravei, extract of Thymus citriodorus, Chrysine, extract of Dianthus carthusianorum or a mixture thereof.

The association of tiliroside/palmitoyl dipeptide 5 diamonohydroxybutyrate offers an enhanced effect when the skin of the mammal, typically a human, is illuminated by the illuminated fabric according to the invention, in particular in the infrared wavelengths.

The cosmetic product may be applied before or after the application of the illuminated fabric to the skin.

When applied before, this makes it possible to increase the effect of the product by the influence of the light emitted by the illuminated fabric according to the invention.

When applied after the application of the illuminated fabric to the skin, this allows for a better absorption of the product by the skin of the user previously illuminated.

Obviously, it is possible to adapt the dimensions of the illuminated fabric according to the invention according to the cutaneous areas to be treated. It is thus possible to produce patches of smaller dimensions than the fabric needed to illuminate a face, in order to treat more targeted areas.

1. An illuminated fabric having a transversal weft direction and a longitudinal warp direction, wherein the web comprises, longitudinally, a plurality of groups of eight consecutive optical fibers interwoven with textile fibers, each group being interwoven transversely according to a mixture of satin 4 weaves, of satin 6 weaves, possibly with floats, and of satin 8 weaves.

2. The illuminated fabric as claimed in claim 1, further comprising additional optical fibers interwoven transversely according to a mixture of incomplete satin 4, satin 6 and satin 8 weaves.

3. The illuminated fabric as claimed in claim 1, in which:
each group is interwoven transversely according to an alternation of patterns A made up of a satin 8 weave and of patterns C made up of four satin 4 weaves arranged in a square, the patterns A and C being systematically separated by a pattern B made up of satin 6 weave, possibly with float; and
two longitudinally successive groups having weaves A and C alternated in opposition.

4. The illuminated fabric as claimed in claim 1, in which an eighth to a quarter of the satin 6 weave consists of float lines in which the optical fibers are not interwoven.

5. The illuminated fabric as claimed in claim 1, in which:
the pattern A has a transversal dimension of between 40 and 50 millimeters,
the pattern B has a transversal dimension of between 25 and 35 millimeters;
the pattern C has a transversal dimension of between 55 and 75 millimeters.

6. The illuminated fabric as claimed in claim 1, having a density of optical fibers of between 29 and 40 optical fibers per cm.

7. The illuminated fabric as claimed in claim 1, in which the optical fibers have a core of PMMA and a sheath of fluorinated polymer.

8. The illuminated fabric as claimed in claim 1, in which the sheath has a thickness of 5 μm, the optical fibers having an overall diameter of 250 μm.

9. The illuminated fabric as claimed in claim 1, in which the textile fibers are made of a material chosen from polyester, cotton, linen, polypropylene, polyamides or a mixture thereof.

10. The illuminated fabric as claimed in claim 1, having a flexibility of less than or equal to 2000 mN, the flexibility being defined as being the force needed to bend a 3.5 cm×5.0 cm sample by 30°.

11. A light-administering device configured to be arranged in contact with the skin of a user, comprising at least one light source and one illuminated fabric as claimed in claim 1, in which at least one end of each optical fiber is connected to a light source.

12. The device as claimed in claim 11, in which the light source has a wavelength of between 440 nm and 680 nm.

13. The device as claimed in claim 11, also comprising a means for storing the duration and/or the frequency of use and a timer capable of stopping or disabling the operation of the light source(s).

14. The device as claimed in claim 11, also comprising a means for reading operating information concerning the device, the reading means being functionally linked with a central processing unit capable of controlling or disabling the operation of the device according to the operating information.

15. A cosmetic skin treatment kit, comprising a device as claimed in claim 11 and at least one cosmetic product container, the container bearing operating information concerning the device.

16. The kit as claimed in claim 15, in which the container comprises an RFID chip on which is stored usage information, the reading means of the device being an RFID chip reader.

17. The kit as claimed in claim 15, in which the container comprises a one- or two-dimensional barcode graphically representing the usage information, the reading means of the device being a one- or two-dimensional barcode reader.

18. A method for implementing a kit as claimed in claim 15, characterized in that the method it comprises the following steps:
implementing the reading means to read the operating information and activate the device in accordance with this information;
directly applying the illuminated fabric to the skin of a mammal for illumino-cosmetic treatment of the skin of the mammal;
a duration and a mode of operation of the light source being automatically controlled according to the operating information.

19. The method as claimed in claim 18, in which the cosmetic product comprises at least one active product taken from the group consisting of: palmitoyl dipeptide 5 diaminohydroxybutyrate, extract of Cochlearia officinalis, a ferment of Thermus thermophillus, tiliroside, pyroglutamylamylamidoethyl indole, a combination of tiliroside/palmitoyl dipeptide 5 diaminohydroxybutyrate, extract of Rhodiola rosea, extract of Spergularia rubra, extract of Camellia sinensis, extract of Lapsana communis, extract of Atractyloides lancea, extract of Cedrelopsis gravei, extract of Thymus citriodorus, Chrysine, extract of Dianthus carthusianorum or a mixture thereof.

20. The method as claimed in claim 19, in which the product is applied before the application of the illuminated fabric to the skin.

21. The method as claimed in claim 19, the cosmetic product is applied after the application of the illuminated fabric to the skin.
